SEQUENCE LISTING (8 sequences)

SEQ ID NO: 1 - protein, 411 aa, Penicillium chrysogenum
MVDPSVSGITKMDTNDIKQNDIPKDQPTLVRWYMDVRRWDEKYFDLPLLETLTQPDQAAVKKYYQTSDKRLSLASQLLKYYYIHQATGTPWSKIEIQRTPMPENRPFYDSSLDFNVSHQAGLTLFAGTRAATAHSLSGGPQTLPRVGIDVACVDEPSRRRANRPPKTLADLATFVDVFSDVLSLRELATIKNPYATLKLARELGLNKSDPSKDDQEVLAAYGIRLFYSIWALKEAYLKMTGDGLLASWIKDLEFTNVVPPEPVQTVGFAGDPSATHAPSVQNWGRPYSDVKISLRGIPDHSVRVQPVGFESDYIVATAASGPNIGSVSRQVVVNDSDHHLPGRITAFDSETGLQNVRIPPIALRSIGDGDPWRVDSKISDPWLPMQEVDIEIDIRPCADGRCEHLRDLPSF

SEQ ID NO: 2 - DNA, 1284 bp, Penicillium chrysogenum
atggtagaccccagtgtgtctggaattgtgagtagccacatagcctccatgagtgcacccactgaccaatttcagaccaaaatggataccaatgatatcaaacagaatgacatccccaaggaccagcccacgttggtccgatggtacatggatgtcagacgttgggatgaaaaatactttgatctccctttgcttgaaaccttaacacagcctgatcaggcagctgtcaagaagtactatcaaacatcggacaagcgcctgtccttggcctcccagttgctgaaatattactacattcaccaagccactggcactccctggagcaagattgagatccagcgtactccgatgcccgaaaatcgaccattctacgattcaagcctggatttcaacgtcagccatcaggctggtctcactctgttcgcaggcacgcgtgccgcaacagcccactccttatccggtggacctcaaacattgcctcgcgtgggaattgacgttgcgtgtgttgatgaaccctctcgtcgtcgtgctaatcgtcccccgaagacacttgccgaccttgcaaccttcgtggatgtcttcagtgacgttctctcactccgtgagcttgcgaccatcaagaacccgtacgcgactcttaaattggctcgtgagcttggtctgaataaaagtgacccgagcaaagacgaccaggaagtccttgctgcctacggcattcggctgttctactcgatttgggctctcaaggaggcttacttgaaaatgaccggagacggccttctggcctcttggataaaggatctggaattcacaaacgttgttccccccgaaccagttcaaacagtcggatttgctggtgatccttctgccactcacgcgccctcggtccaaaattggggccggccttactccgatgtcaaaatctccttgcgtggcattcctgaccattctgtgcgcgttcagctcgtcggcttcgagtccgactacatagttgccacggccgcgtcgggccccaatattggatccgtttcgcggcaggtagtcgtgaatgacagcgatcaccatctgccagggcgtatcacagccttcgactctgagactggactccagaacgtccgcattcccccaatcgcgcttcgatcaattggcgatggggacccctggcgtgtggactcgaaaatcagcgacccctggctccccatgcaggaggtcgatattgaaatcgatatccggccctgtgcggatggtcgttgcgagcacctacgggatttaccaagcttttaa

SEQ ID NO: 3 - DNA, 1236 bp, Penicillium chrysogenum
atggtagaccccagtgtgtctggaattaccaaaatggataccaatgatatcaaacagaatgacatccccaaggaccagcccacgttggtccgatggtacatggatgtcagacgttgggatgaaaaatactttgatctccctttgcttgaaaccttaacacagcctgatcaggcagctgtcaagaagtactatcaaacatcggacaagcgcctgtccttggcctcccagttgctgaaatattactacattcaccaagccactggcactccctggagcaagattgagatccagcgtactccgatgcccgaaaatcgaccattctacgattcaagcctggatttcaacgtcagccatcaggctggtctcactctgttcgcaggcacgcgtgccgcaacagcccactccttatccggtggacctcaaacattgcctcgcgtgggaattgacgttgcgtgtgttgatgaaccctctcgtcgtcgtgctaatcgtcccccgaagacacttgccgaccttgcaaccttcgtggatgtcttcagtgacgttctctcactccgtgagcttgcgaccatcaagaacccgtacgcgactcttaaattggctcgtgagcttggtctgaataaaagtgacccgagcaaagacgaccaggaagtccttgctgcctacggcattcggctgttctactcgatttgggctctcaaggaggcttacttgaaaatgaccggagacggccttctggcctcttggataaaggatctggaattcacaaacgttgttccccccgaaccagttcaaacagtcggatttgctggtgatccttctgccactcacgcgccctcggtccaaaattggggccggccttactccgatgtcaaaatctccttgcgtggcattcctgaccattctgtgcgcgttcagcccgtcggcttcgagtccgactacatagttgccacggccgcgtcgggccccaatattggatccgtttcgcggcaggtagtcgtgaatgacagcgatcaccatctgccagggcgtatcacagccttcgactctgagactggactccagaacgtccgcattcccccaatcgcgcttcgatcaattggcgatggggacccctggcgtgtggactcgaaaatcagcgacccctggctccccatgcaggaggtcgatattgaaatcgatatccggccctgtgcggatggtcgttgcgagcacctacgggatttaccaagcttttaa

SEQ ID NO: 4 - DNA, 3188 bp, Penicillium chrysogenum
gtcgaccgaagtggtttcggttcactcgcacatcaagaccaccgatcagctcttgcccgcccttctttgtcttgttggcagactcggcaagcaaaatgagcccggcgcatgtaccccacgtcggtttgcgatccactctgcataacccacgtattagatcgaattgatatggactaacccggttcactcactttacgaattctcgcagtggctcgagaagatttgaccttgctgcgactaaagacatagtggtactctcgcctccgggcaagaccaggccgtcgcatgttgccagttcttgtggcgtccgtacttcaatgaagtgccattccgacggctgcgcttgctcagcggcctttttcaaaagctgcacatgctcaaagaatgcgccctgtagggccaggactccaacagtgatagccatttcctctgaagatcggaattgcggaccctccgagctcgggtgcttcttgatattgatgactctttttaaagcacatgactttgactttccggcggggaacgtatcaacacgtgatggcggcttatctccatctttaattccacgcgacatcaggatatcgtgagagctctcggacgattcctgcgcactttgaaaacagactgcataaccgaggcattatagtataaaacaaatagactcacctacagaaagagtgataagttaggtcctatacctgtttccaatgtttctctctcttgctggatcagctttaacatatctatggatggtatcttggatagtcatagtcatattgcgcttgctattgcatgtctctttgctacatcctatttatggtattatgtacacggcctgtttctcgtttgccggcctattgatgtatacatgtattggtgtaggtagttattgcctcgccttatcgacacgtgctgatagataaggaccccgataagacgccaacatggcttctatccaggtgtggatgctccgcatccaaggtgcgaatatacgagatcacaatgcaatggtagaccccagtgtgtctggaattgtgagtagccacatagcctccatgagtgcacccactgaccaatttcagaccaaaatggataccaatgatatcaaacagaatgacatccccaaggaccagcccacgttggtccgatggtacatggatgtcagacgttgggatgaaaaatactttgatctccctttgcttgaaaccttaacacagcctgatcaggcagctgtcaagaagtactatcaaacatcggacaagcgcctgtccttggcctcccagttgctgaaatattactacattcaccaagccactggcactccctggagcaagattgagatccagcgtactccgatgcccgaaaatcgaccattctacgattcaagcctggatttcaacgtcagccatcaggctggtctcactctgttcgcaggcacgcgtgccgcaacagcccactccttatccggtggacctcaaacattgcctcgcgtgggaattgacgttgcgtgtgttgatgaaccctctcgtcgtcgtgctaatcgtcccccgaagacacttgccgaccttgcaaccttcgtggatgtcttcagtgacgttctctcactccgtgagcttgcgaccatcaagaacccgtacgcgactcttaaattggctcgtgagcttggtctgaataaaagtgacccgagcaaagacgaccaggaagtccttgctgcctacggcattcggctgttctactcgatttgggctctcaaggaggcttacttgaaaatgaccggagacggccttctggcctcttggataaaggatctggaattcacaaacgttgttccccccgaaccagttcaaacagtcggatttgctggtgatccttctgccactcacgcgccctcggtccaaaattggggccggccttactccgatgtcaaaatctccttgcgtggcattcctgaccattctgtgcgcgttcagctcgtcggcttcgagtccgactacatagttgccacggccgcgtcgggccccaatattggatccgtttcgcggcaggtagtcgtgaatgacagcgatcaccatctgccagggcgtatcacagccttcgactctgagactggactccagaacgtccgcattcccccaatcgcgcttcgatcaattggcgatggggacccctggcgtgtggactcgaaaatcagcgacccctggctccccatgcaggaggtcgatattgaaatcgatatccggccctgtgcggatggtcgttgcgagcacctacgggatttaccaagcttttaaattccttcttgctgggatatgaccaggcgaccatgcacccgagttatttgcatattgcatctcctcatctcatattcctttctgagcgtgtttttcggagcgataattacccttgaacatatttctgcattgctgtattgccattagcgaaaattcccgagctagttgtagttgatttcctggaacgctgggggagtgccgctcagatgttcatctccaataagcccctcaatgaatcttcacttcatcggatccaaggtcaatcttcgagatcaagtgcaagttgcccagaaagcacgggtaaagaaaccaagcctatttctattctatggtctaatgtaaactaaaaatgtagaaggaagaaaagcaagtatccaacagtaggcgggtcatgacatgcgtgtgcgctaaggatatatacatttcgaattgcaaagagggaagaggtgaatcaggagtgaaatgtgtgtcaagaggcaatgtcaatgtcaagatcattgttgctctcatgagcagtcacggattgtgtcggattgttcggcgtctggggccctcagattctatttctgggtcatgagcttgagagtaggtaccgaagaagtgagcagtattatactgcagtgagtgtttagggggaattccttctggtgaattgtggcgttcggggttgctctccggtcttatgggtcttaatctggatgcccgatagtgcacccaagttaggagaaaaacatatggtaagtgttaatcgtggagcagtgtggcgaatcgcgaattgggtttggcacttagatttcgatggcgctagagacgccgttggcgcgagcaccatcgacctcatttttatgcgcgtgggacattgctgcaagagttttgagcatcgaatcccgcgtcgac

SEQ ID NO: 5 - DNA, 37 bp, Artificial Sequence
  oligonucleotide primer
ccccgtcgaccgaagtggtttcggttcactcgcacat

SEQ ID NO: 6 - DNA, 37 bp, Artificial Sequence
  oligonucleotide primer
ccccgtcgacgcgggattcgatgctcaaaactcttgc

SEQ ID NO: 7 - DNA, 41 bp, Artificial Sequence
  oligonucleotide primer
gggggccgaggcggcccatggataccaatgatatcaaacag

SEQ ID NO: 8 - DNA, 43 bp, Artificial Sequence
  oligonucleotide primer
gggggccattatggcctcattcaggactacctgccgcgaaacg

(19) United States
(12) Patent Application Publication
(10) Pub. No.: US 2007/0042457 A1
(43) Pub. Date: Feb. 22, 2007

(54) Process for producing penicillin

(76) Inventors: Kurt Schoergendorfer, Langkampken (AT); Hubert Kurnsteiner, Angerberg (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/575,560

(22) PCT Filed: Oct. 14, 2004
(86) PCT No.: PCT/EP04/11566

§ 371 Date: Sep. 11, 2006

(30) Foreign Application Priority Data
Oct. 15, 2003 (AT) 1622/2003

(51) Int. Cl. C12P 37/00; C07H 21/04; C12N 9/10; C12N 1/16; C12N 15/74

(52) U.S. Cl. 435/43; 435/193; 435/254.3; 435/254.5; 536/23.2; 435/484

(57) ABSTRACT

The invention relates to isolated nucleic acid molecules, which code for a new protein of Penicillium chrysogenum, vectors which comprise a nucleic acid molecule of this kind, host cells which are transformed with such a nucleic acid molecule or vector, and a process for the production of penicillin using such transformed host cells.

### FIELD OF THE INVENTION

The invention relates to isolated nucleic acid molecules, which code for a new protein of Penicillium chrysogenum, vectors which comprise a nucleic acid molecule of this kind, host cells which are transformed with such a nucleic acid molecule or vector, and a process for the production of penicillin using such transformed cells.

### BACKGROUND TO THE INVENTION

Penicillin is a natural metabolite, which is obtained on an industrial scale by fermentation of the filamentous fungus Penicillium chrysogenum (hereinafter referred to as P. chrysogenum). In addition, Penicillin G and Penicillin V form important precursors for a series of semi-synthetic penicillin antibiotics. The class of penicillin substances is of great therapeutic significance. Increases in the yield of industrial penicillin fermentations are based not only on technological improvements in the process, but also essentially on a continuous improvement of the genetic strain. In the foreground of modern strain-improvement methods is always the transformation of production strains with specific genes that have a potential to increase production. For a small group of known penicillin-biosynthesis genes, a strain-improvement potential may be assumed based on the understanding of biochemical relationships of penicillin biosynthesis. Amplification, i.e. an increase in the number of copies of such known genes, partly shows in an experiment an actual significant improvement in productivity of a production organism. However, the group of known genes, for which a strain improvement potential can be predicted from the point of view of scientific plausibility considerations, is very small. As well as these known biosynthesis genes, an unknown number of further genes can be assumed to similarly effect production-increasing potential by means of amplification. The function of such genes is frequently unknown, since all the cellular processes that have an influence on penicillin biosynthesis are barely understood at the present time. Strategies for identifying further genes with production-increasing potential are therefore of great significance.

The important penicillin biosynthesis genes, ACV synthetase (ACVS), isopenicillin-N (IPN) synthase and the acyl CoA: IPN acyl transferase have already been known for a long time. The central enzyme is the ACVS, a non-ribosomal peptide synthetase (NRPS), which catalyses the formation of the tripeptide ACV. It was only known in recent years that, for a few microorganisms, NRPS must be “charged” with phosphopantethein in order to be brought to an active form.

4′-Phosphopantethein transferases (PPTases) catalyst the transfer of the 4′-phosphopantethein group (Ppant) from coenzyme A (CoASH) to the hydroxyl function of the side chain of a preserved serine residue, which is found in Ppant-dependent carrier proteins. The carrier protein, generally abbreviated to XCP, is thus converted from the catalytically inactive apo-form to the catalytically active holo-form. The reaction is Mg2+ dependent and forms 3′-5′-ADP as a by-product. There are various Ppant-dependent biosynthesis routes. In each cell there is fatty acid biosynthesis, in which the acyl-carrier protein (ACP) binds the intermediates. Many antibiotics and natural substances, such as cyclosporin and the β-lactams, are produced by non-ribosomal peptide synthetases (NRPS) or polyketide synthases (PKS), which contain peptidyl-carrier proteins (PCP) or ACP. Finally, a specialised peptide synthetase is to be found, in a biosynthesis route leading to lysine, in fungi and a few plants. What is common to all these biosynthesis routes is that the participating carrier proteins are phosphopantetheinylated by PPTases and thereby converted into the active form. The PPTases are essential factors for these processes, however the genes coding for them are still unknown in many cases. In P. chrysogenum, such a PPTase or the corresponding gene has not been described up to now. The discovery of such a previously unknown gene of P. chrysogenum, which codes for a PPTase, thus forms a central aim of the present invention. It is therefore an aim of the present invention to prepare a nucleic acid as well as vectors, which code for a new protein of P. chrysogenum, and may be used for transformation of a P. chrysogenum host cell, so that this host cell is in a position to deliver penicillin in good yields. A further aim of the present invention is to prepare a transformed host cell of this kind. Finally, it is a further aim of the present invention to prepare a process for the production of penicillin, using the said transformed host cell.

### FIGURES

FIG. 1 shows the amino acid sequence (SEQ ID NO 1=sequence identity no 1) of a new protein of P. chrysogenum, which is derived from the nucleic acid molecules according to the invention (nucleic acid sequence according to FIG. 2 or 4). The illustration is from the N-terminus to the C-terminus.

FIG. 2 (SEQ ID NO 2) shows the genomic DNA sequence including the 1 intron of the coding region of the ppta gene of P. chrysogenum from the translation start codon (ATG) to the translation stop codon (TAA). The intron is underscored, a single strand from the 5′- to 3′-direction is illustrated.

FIG. 3 (SEQ ID NO 3) shows the cDNA sequence of the coding region of the new gene from the translation start codon (ATG) to the translation stop codon (TAA); a single strand from the 5′- to 3′-direction is illustrated.

FIG. 4 (SEQ ID NO 4) shows the genomic DNA sequence of a Sall fragment of a genomic clone of the new gene (a single strand from the 5′- to 3′-direction is illustrated). The translation start codon (ATG) and the translation stop codon (TAA) of the coding region are underscored and illustrated in bold; the intron is underscored.

### DETAILED DESCRIPTION OF THE INVENTION

In the context of the present invention, a new gene of P. chrysogenum is described, which codes for a previously unknown protein in P. chrysogenum. It is shown that this is a new PPTase, and that strains with high penicillin titres for an industrial scale can result from co-transformation experiments with this gene.

The new gene can be isolated from the P. chrysogenum strain P2=ATCC 48271 (obtainable under this number from the ATCC, American Type Culture Collection, P.O. Box 1549, Manassas, Va. 20108, USA) The new gene may also be found, however, in other strains of P. chrysogenum. Alternatively, nucleic acid and amino acid sequences or molecules depicted here may also be produced artificially.

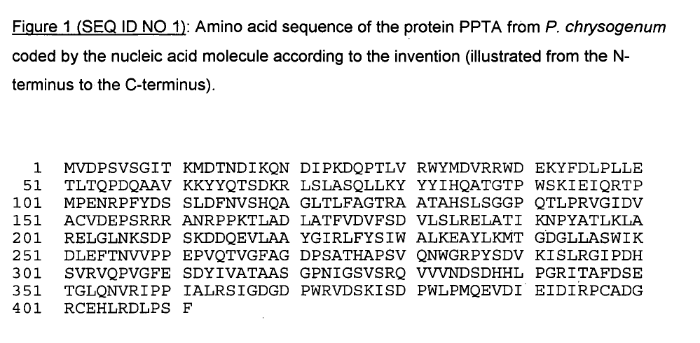

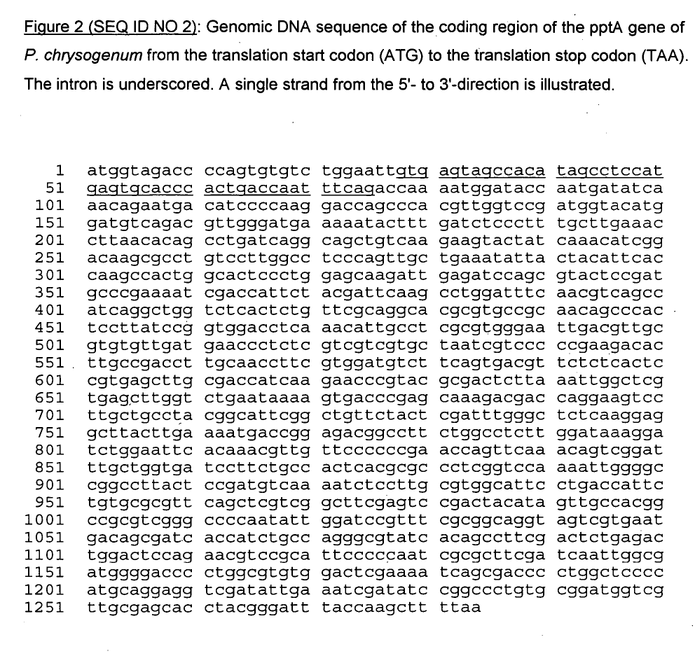

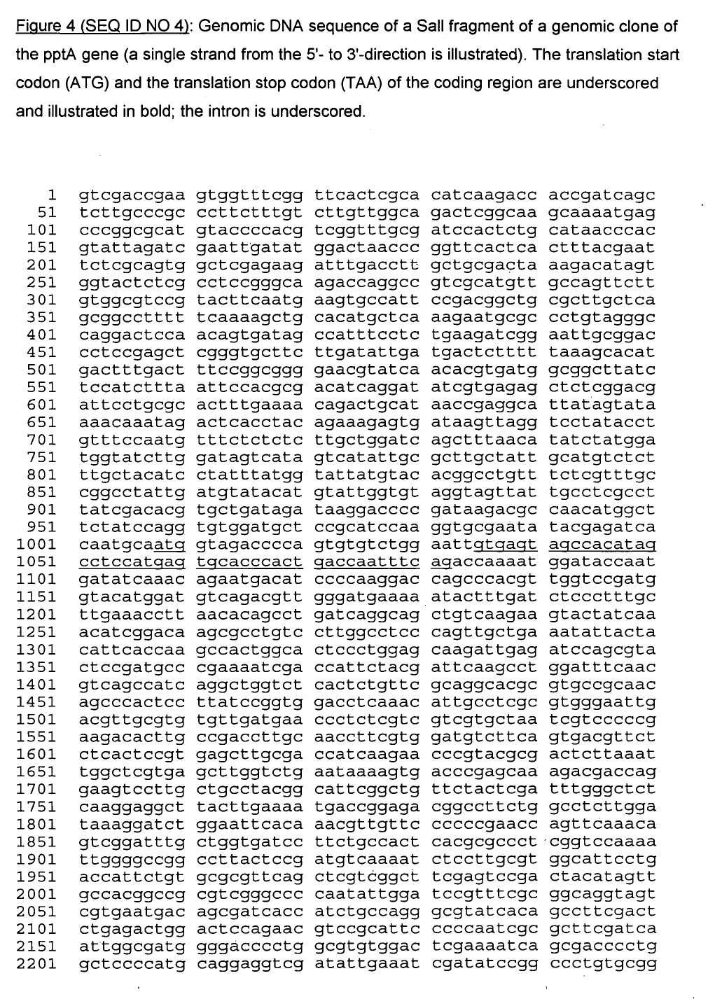

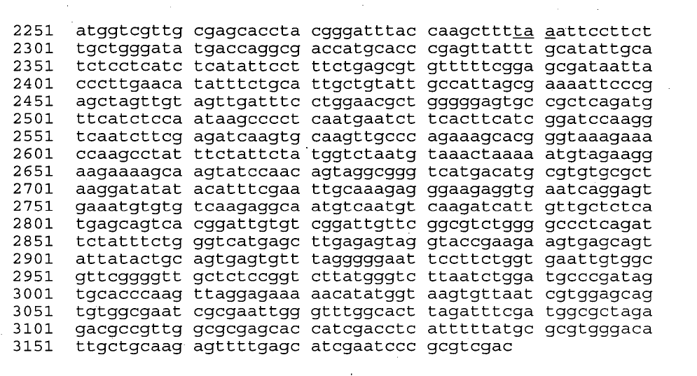

The gene codes for a protein with a length of 411 amino acids. The amino acid sequence is illustrated in FIG. 1. In the gene, the coding region is interrupted by 1 intron, as can be seen in FIGS. 2 and 4.

Based on functional tests (see example 2), the gene of P. chrysogenum according to the invention is characterised as the gene for a previously unknown PPTase and is called a ppta gene.

An object of the present invention is therefore an isolated nucleic acid molecule, which codes for a protein that includes the amino acid sequence according to SEQ ID NO 1.

A nucleuc acid molecule of this kind can thus code, for example, for a protein which contains further amino acids in addition to the listed amino acid sequence (SEQ ID NO 1), for example for a fusion protein. If desired, such fusion proteins can play the role of producing the new protein in isolated form. The fusion sections can, for example, increase stability or simplify purification.

A nucleic acid molecule according to the invention, which only codes for an amino acid sequence according to SEQ ID no 1, is preferred in the context of the present invention. Such a nucleic acid molecule may advantageously be used for the purpose of the production of penicillins described below, especially penicillin V or G. A further object of the present invention is therefore a nucleic acid molecule according to the invention, which codes for a protein which has exclusively the amino acid sequence according to SEQ ID NO 1.

A nucleic acid molecule according to the invention is preferably a DNA molecule. Alternatively, the nucleic acid molecule may be a RNA molecule, especially a mRNA molecule.

A DNA molecule according to the invention may be prepared, for example, by producing a genomic DNA library of the genome of the said P. chrysogenum strain ATCC48271. A genomic clone is identified by “screening” with homologous probes, the structures of which may be derived from the described nucleic acid sequence of the gene according to FIG. 4. Corresponding techniques are known from literature (e.g. in T. Maniatis et al., Molecular Cloning—A Laboratory Manual, 1982, Cold Spring Harbor Laboratory, Cold Spring Harbor, N.Y., USA). The desired DNA molecule is found on a 3.2 kb Sall fragment of such a clone, which can be isolated or prepared by classic techniques. Such a fragment is illustrated in FIG. 4. A preferred embodiment of the present invention thereby relates to a nucleic acid molecule according to the invention, comprising a base sequence according to SEQ ID NO 4 or a base sequence which only differs from the sequence according to SEQ ID NO 4 on the basis of the degeneration of the genetic code. This means, according to the invention, that objects of the invention are also those nucleic acid molecules that differ from the specifically listed sequences, in that one or more of the listed codons are replaced by one or more others, in such a way that the amino acid sequence of the coded protein (SEQ ID NO 1) is not changed. This includes the use of one (or more) alternative stop codon(s). This also applies to the other nucleic acid molecules described below. The nucleic acid molecule according to SEQ ID NO 4 contains regulatory sequences (such as a promoter and a stop codon) and may advantageously be used for transformation, especially in a vector, of P. chrysogenum, and thus for the production of penicillin, especially penicillin G or penicillin V.

The said 3.2 kb Sall fragment comprises especially the coding part of the new gene. This part is illustrated in FIG. 2 and comprises 1 intron. A further embodiment of the present invention is therefore a nucleic acid molecule according to the invention, comprising a base sequence according to SEQ ID NO 2 or a base sequence which only differs from the sequence according to SEQ ID NO 2 on the basis of the degeneration of the genetic code, as explained above. Such a nucleic acid molecule thereby corresponds to the genomic DNA sequence of the coding part of the new gene. Further preferred embodiments of the present invention are those nucleic acid molecules that are distinguished from the SEQ ID NO 2 by the absence of an intron.

Therefore, a nucleic acid molecule according to the invention, comprising a base sequence according to SEQ ID NO 3 or a base sequence which only differs from the sequence according to SEQ ID NO 3 on the basis of the degeneration of the genetic code, as explained above, is further preferred. A nucleic acid molecule of this kind no longer comprises an intron and is to be equated as such to a corresponding cDNA sequence.

In addition, the production of a nucleic acid molecule according to the invention (including a said cDNA molecule) can take place, for example, fully artificially or semi-artificially. RNA or mRNA molecules according to the invention may be isolated by standard techniques from the microorganism P. chrysogenum or produced artificially. It is possible to produce a corresponding cDNA molecule from a corresponding mRNA by standard techniques.

Whereas the said nucleic acid molecules may contain further base sequences throughout (in order e.g. to code for a fusion protein), preferred embodiments relate to a nucleic acid molecule according to the invention, which has exclusively one base sequence, which is selected from the group of base sequences SEQ ID NO 2, SEQ ID NO 3, SEQ ID NO 4 and a base sequence that only differs from one of the said sequences on the basis of the degeneration of the genetic code, as explained above.

In a further embodiment, nucleic acid molecules according to the invention additionally contain one or more stop codons at their C-terminus directly after the end of the coding region. The naturally occurring stop codon, which has been identified at TAA, is preferred. Alternatively, however, the other known stop codons may also be used. The use of several stop codons is also possible.

A further object of the present invention relates to a vector which comprises one of the mentioned nucleic acid molecules according to the invention. Such a vector is preferably suitable for transformation of a host cell. In particular, a host cell of this kind is a microorganism. Preferably, such a microorganism is a filamentous fungus. The filamentous fungus is advantageously selected from the group consisting of Penicillium chrysogenum, Penicillium notatum, Penicillium brevicompactum, Penicilium citrinum, Acremonium chrysogenum, Aspergillus nidulans, Aspergillus niger, Aspergillus fumigatus, Aspergillus terreus and Tolypocladium inflatum In a particularly preferred embodiment of the present invention, the microorganism or the filamentous fungus is Penicillium chrysogenum.

Such a vector may exist, for example, in the form of a plasmid. In addition to a nucleic acid molecule according to the invention, this kind of vector contains further sequences where necessary, e.g. a replication source and further regulatory elements (promoter, translation start signal and termination signal, etc.), so that after transformation has taken place, expression of the nucleic acid molecule according to the invention may be effected. After transformation has taken place, a nucleic acid molecule according to the invention, as well as further vector elements, can be integrated into the genome of the host cell, which corresponds to an amplification of the coding part of the new gene. A vector according to the invention advantageously comprises a nucleic acid molecule which contains a base sequence according to SEQ ID NO 4. Such a base sequence corresponds to the said Sall fragment and already contains regulatory sequences, for example a corresponding promoter.

Such vectors may be produced by standard techniques by cloning a nucleic acid molecule into appropriate standard vectors.

A further object of the present invention relates to a host cell, which is transformed with a nucleic acid molecule according to the invention or with a vector according to the invention. In particular, a host cell of this kind is a microorganism. Preferably, such a microorganism is a filamentous fungus. The filamentous fungus is advantageously selected from the group consisting of Penicillium chrysogenum, Penicillium notatum, Penicillium brevicompactum, Penicilium citrinum, Acremonium chrysogenum, Aspergillus nidulans, Aspergillus niger, Aspergillus fumigatus, Aspergillus terreus and Tolypocladium inflatum. In a particularly preferred embodiment of the present invention, the host cell (or the microorganism or the filamentous fungus) is Penicillium chrysogenum.

Transformation of such a host cell, especially of P. chrysogenum, with a vector according to the invention is effected according to standard processes. Such a process is described for example in Austrian patent specification AT 391 481, examples 6, 8, 10 and 12.

Alternatively, a so-called co-transformation may also be carried out. In this case, the vector with a selection marker and the vector with the gene according to the invention are used as separate molecules in the transformation.

Alternatively, the nucleic acid molecules according to the invention may also be used for transformation.

In particular, the genes to be inserted (the gene according to the invention and at least one marker gene) may themselves be used separately, as linear nucleic acid molecules. A certain proportion of transformed host cells, which carry the vector for selectioning or the corresponding selection gene, then also contains the second gene used in such co-transformation. The proportion is dependent on the individual experiment and on the selected practical test parameters.

A transformed P. chrysogenum host cell according to the invention may advantageously be used to produce penicillin. A further object of the present invention therefore relates to a process for the production of penicillin, comprising the cultivation of a P. chrysogenum host cell according to the invention under conditions that are appropriate for effecting the formation of penicillin with the host cell. The penicillin is most preferably selected from the group consisting of penicillin G and penicillin V.

Suitable cultivation/fermentation techniques are known to the specialist in the field of antibiotics and have been used for a long time for the production of penicillins.

In a preferred embodiment, the process according to the invention also includes the isolation of the penicillin formed. The penicillin formed from a transformed P. chrysogenum host cell according to the invention may be purified and isolated from the fermented mycelium paste by known techniques, for example extraction with butyl acetate and subsequent chromatography.

Penicillin produced according to the invention, especially penicillin G or penicillin V, may be preferably reacted to further derivatives having antibiotic properties.

An alternative application of the present invention concerns an isolated protein, which contains an amino acid sequence according to SEQ ID NO 1. As mentioned, such a protein also contains corresponding fusion proteins, from which, where desired, a mature protein with an amino acid sequence according to SEQ ID NO 1 can be produced by cleavage. Preference is given to a protein according to the invention, in which the protein has exclusively the amino acid sequence according to SEQ ID NO 1.

A protein according to the invention may be produced whereby an appropriate procaryotic or eucaryotic host cell, which contains an appropriate expression vector according to the invention, which comprises a nucleic acid molecule coding for the protein, is cultivated under conditions that effect expression of the protein. The protein may be purified and isolated by conventional techniques. Suitable procaryotic host cells, in which a cDNA according to the invention is used in particular, are for example bacterial cells, e.g. E. coli; suitable eucaryotic host cells are, for example, yeast cells, such as Saccharomyces cerevisiae or Pichia pastoris, or mammalian cells, such as CHO or BHK cells.

A protein according to the invention may be used for example in order to bring corresponding enzymes, such as NRPS or PKS, or individual module or domain units thereof, with a 4′-phosphopantethein group in vitro from the apo-form to the enzymatically active holo-form (see above). The protein according to the invention is therefore a valuable tool for producing active in vitro systems for producing new molecules, such as systems for combinatory biosynthesis, from representatives of the said enzyme groups (such as NRPS or PKS), but also from other 4′-phosphopantathein-containing enzymes, or individual parts thereof.

Where references are made, these are incorporated insofar as necessary.

The present invention is explained more fully by the following examples, but is not restricted to them. In particular, the examples relate to preferred embodiments of the present invention.

### EXAMPLES

The materials and reagents mentioned herein are familiar to the person skilled in the art, are available commercially or are readily obtainable, and may be used in accordance with the manufacturer's instructions.

### Example 1

### Isolation of the New Gene ppta from Penicillium chrysogenum

The gene according to the invention is produced using the polymerase chain reaction. Here, DNA is isolated from the Penicillium chrysogenum strain ATCC48271. Cells of the fungus are broken up mechanically in liquid nitrogen by trituration in a mortar, and subsequently isolated by standard techniques, such as that described by T. Maniatis et al., Molecular Cloning—A Laboratory Manual, 1982, Cold Spring Harbor Laboratory, Cold Spring Harbor, N.Y., USA, or using a commercially available kit, such as that of the company Qiagen.

With the aid of the primers PCR1f and PCR1 r, under standard conditions with a heat-stable DNA polymerase, a ca. 3.2 kb amplificate is produced from the genomic DNA.

Primer PCR1f

(SEQ ID NO 5)

5′-CCCCGTCGACCGAAGTGGTTTCGGTTCACTCGCACAT
Primer PCR1r

(SEQ ID NO 6)

5′-CCCCGTCGACGCGGGATTCGATGCTCAAAACTCTTGC

For cloning the amplified region, which corresponds to the nucleic acid molecules according to the invention according to SEQ ID NO 4, the fragment with the restriction endonuclease Sail is cleaved and ligated into a E. coli plasmid used as a standard via a Sall site; a standard plasmid of this type is, for example pBluescript II SK+ (Stratagene).

The ligation product is transformed into E. coli (e.g. strain DH5alpha) and produced there in a quantity sufficient for the other steps, and purified. Depending on the method of construction, plasmids may also result, which contain the nucleic acid molecule in a reversed orientation; these structures are however basically of the same funcionality.

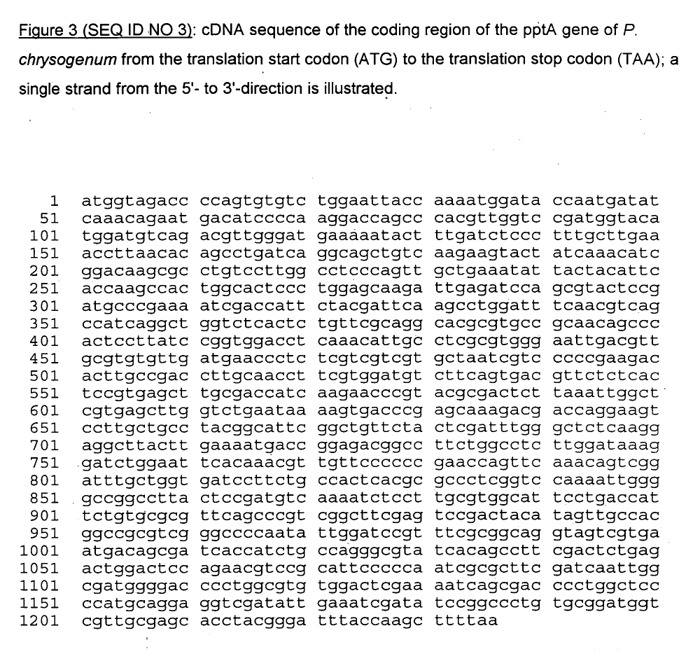

The subsequent sequencing and evaluation is given by the nucleic acid sequences illustrated in FIGS. 2 (SEQ ID NO 2) and 4 (SEQ ID NO 4). A cDNA sequence according to FIG. 3 (SEQ ID NO 3) may then be derived therefrom, as can the amino acid sequence of the coded protein according to FIG. 1 (SEQ ID NO 1). Basically, a conclusive verification of the cloning product may be effected by sequencing and by a sequence comparison with the DNA sequences illustrated in FIGS. 2 and 4. A plasmid which bears the Sall fragment is called plasmid1 and is used in the following.

### Example 2

### Functional Characterisation of the New Gene ppta from Penicillium chrysogenum

For functional characterisation of the new gene, the cDNA of the new ppta gene from Penicillium chrysogenum is amplified by PCR from whole cDNA of P. chrysogenum. The whole cDNA is produced from mRNA of P. chrysogenum using commercially available kits (e.g. by Qiagen) and standard laboratory methods.

Using primers PCR2f and PCR2r, a ca. 1.25 kb amplificate is produced from the cDNA and incorporated into the yeast vector pYES2.1-Sfi.

Primer PCR2f

(SEQ ID NO 7)

5′-GGGGGCCGAGGCGGCCCATGGATACCAATGATATCAAACAG
Primer PCR2r

(SEQ ID NO 6)

5′-GGGGGCCATTATGGCCTCATTCAGGACTACCTGCCGCGAAACG

The vector and further implementation of the functional tests are described in detail in H. D. Mootz et al., “Functional characterization of 4′-phosphopantetheinyl transferase genes of bacterial and fungal origin by complementation of Saccharomyces cerevisiae lys5”, FEMS Microbiol. Lett. 213 (2002), pp. 51-57. The yeast expression vector used in the present example for the functional test is produced analogously to pYES2-npgA (H. D. Motz et al., see above, chapter 2.2, page 53).

The test is based on the functional complementation of a specific defect in a yeast strain. The Lys5-gene codes for a PPTase, which is essential to produce the amino acid lysine in the yeast cell, and thus enables the yeast cell to grow on minimal medium without lysine. A specially constructed yeast strain, in which the Lys5 gene has been destroyed, can no longer produce lysine. The gene according to the invention, incorporated into the above-mentioned yeast-expression vector, is transformed into this yeast strain. The test is described in detail in chapter 3, pages 54-55 of the said publication by H. D. Mootz et al., see above.

Corresponding yeast transformants with the expressed ppta gene from P. chrysogenum can be grown again on the selection medium (minimal medium without lysine), the lys5 defect is complemented (see also chapter 3.4, page 55 of the said publication by H. D. Mootz et al.). This therefore shows that the ppta gene from P. chrysogenum is a gene for a functional 4′-phosphopantetheinyl transferase.

### Example 3

### Co-Transformation of Penicillium chrysogenum

The nucleic acid molecule according to the invention described in example 1 is prepared from a corresponding amount (depending on the number of transformation assays to be carried out) of plasmid1 by restriction with Sall and subsequent purification of the 3.2 kb fragment by means of agarose gel electrophoresis, and it is prepared for transformation.

As a selection marker, the niaD-gene from P. chrysogenum is used in the form of a part fragment of the plasmid J-12 described in Austrian patent specification AT 391 481. To do this, the plasmid J-12 is cleaved with EcoRI and the ca. 6.5 kb fragment, which carries the niaD fragment, is ligated into the plasmid pUCBM20 (Roche Diagnostics) linearised with EcoRI. The result is two possible plasmids, which are each of ca. 9.1 kb, and which differ in the orientation of the inserted EcoRI fragment. Corresponding plasmids of subclones are investigated with a common digestion of the enzymes Xmal and Agel. A clone with the orientation, at which a ca. 2 kg and a ca. 7.1 kb fragment are obtained, is selected and called p1649A. The plasmid p1649A is cleaved with Xmal and Agel and the ca. 7.1 kb fragment is religated, since Xmal and Agel have compatible ends. Plasmids from corresponding E. coli clones are tested by restriction with EcoRI and called p1649C.

For transformation of P. chrysogenum protoplasts of the corresponding strains, a linear part fragment of the plasmid p1649C is used, namely the ca. 4.5 kb EcoRI/XbaI fragment. This is produced by restriction with the enzymes EcoRI and XbaI and subsequent preparation. The fragment carries the niaD gene. Of course, the complete plasmids p1649C, p1649A or J-12 can similarly be used accordingly for transformation.

In principle, all P. chrysogenum strains which are available for an appropriate selection system can be used as recipient strains for transformation. The two produced fragments, which accordingly contain the new gene and the niaD marker, are transformed into a P. chrysogenum strain (PC-180060), which is characterised as niaD mutant, by means of a standard procedure for protoplast transformation. Alternatively, a commercially available P. chrysogenum strain such as ATCC48271 (called P. chrysogenum strain P2) is used for transformation.

The protoplast transformation method used is described e.g. in Austrian patent specification AT 391 481 (see in particular examples 6, 8, 10 and 12) and includes the generation of a nitrate reductase mutant, transformation thereof and subsequently a selection of transformants for nitrate-containing nutrient agar. The properties of the niaD gene used for this selection are likewise described in the indicated reference.

A protoplast density of 10⁸/ml in KCM buffer (0.7 M KCl/50 mM CaCl2/10 mM MOPS/pH 5.8) is set. The aliquots of the DNA solutions of the DNA fragments to be transformed are added to 100 μl of this suspension, whereby the added volume is 10 μl. The ratio of the two fragments is selected in a molar ratio of 1-1.5:1, but can of course be added in another ratio. Ca. 1.5-3.5 μg of the ca. 4.5 kb EcoRI/XbaI fragment (containing the niaD gene) and ca. 0.8-1.8 μg of the ca. 3.2 kb Sall fragment of example 1 (containing the gene according to the invention) are added per transformation assay. Subsequently, 50 μl of PEG (polyethylene glycol) solution (50 mM CaCl₂, 10 mM tris pH7.5, 20% PEG) are added, mixed and incubated for 20 mins on ice. A further 0.5 ml of PEG solution (see above) are added, carefully mixed by rotating the test tube and left to stand for 5 minutes at room temperature. Afterwards, 1.5 ml of KCM buffer are added and mixing again carefully takes place. Finally, about half of the transformation assay is mixed with 7 ml of R1 soft agar and poured onto the selection agar R1 (see also Austrian patent specification AT 391 481). After incubation of the agar plates for about two weeks at 25° C., the colonies of transformants were grown on well and could be used further.

Transformants from such experiments are tested, for example, by Southern hybridization for the presence of additionally integrated copies of the gene according to the invention, or of the essential part of plasmid1 employed.

### Example 4

### Production of Penicillin

Transformants produced in example 3 are tested for penicillin production in fermentation tests in a flask. It is appropriate to compare, in parallel, a population of the same size of ca. 500-1000 co-transformants and transformants. To do this, supernatants of these flask fermentations are evaluated by HPLC analysis.

A corresponding process for penicillin G or V, depending on whether phenyl acetate or phenoxy acetate was added as precursor, is described, for example, in C. S. Ho et al., “Enhancing Penicillin Fermentations by Increased Oxygen Solubility Through the Addition of n-Hexadecane”, Biotechnology and Bioengineering 36 (1990), pp. 1110-1118.

The penicillin titre in the flask fermentations can be determined by HPLC analysis, approximately as in L. H. Christensen et al., “A robust liquid chromatographic method for measurement of medium components during penicillin fermentations”, Analytica Chimica Acta 296 (1994), pp. 51-62.

In order to obtain statistically relevant amounts of data, these analyses are repeated several times (e.g. 6 times), and for each repetition, several (e.g. 4) parallel flask fermentations are carried out on each strain, and tested individually. In this way, from the P. chrysogenum strain PC-180060, three strains were identified (PC-20494, PC-20495 and PC-20496) which stem from the co-transformation with the ppta gene strain according to the invention and have considerably higher penicillin productivity than the starting strain. These strains may be used for production purposes for penicillins, especially penicillin G or penicillin V, on an industrial scale.

The penicillin produced by the transformed strains is extracted from the fermentation broth and purified by standard methods (e.g. according to A. H. Rose (Ed.), Secondary Products of Metabolism, Academic Press London, 1978, pages 75-86).

1. An isolated nucleic acid molecule which encodes a protein which comprises the amino acid sequence as depicted in SEQ ID No. 1.

2. A nucleic acid molecule according to claim 1 which encodes a protein which only possesses the amino acid sequence as depicted in SEQ ID No. 1.

3. A nucleic acid molecule according to claim 1 which is a DNA molecule.

4. A nucleic acid molecule according to claim 3 which comprises a base sequence as depicted in SEQ ID No. 2 or a base sequence which only differs from the sequence as depicted in SEQ ID No. 2 because of the degeneracy of the genetic code.

5. A nucleic acid molecule according to claim 3 which comprises a base sequence as depicted in SEQ ID No. 3 or a base sequence which only differs from the sequence as depicted in SEQ ID No. 3 because of the degeneracy of the genetic code.

6. A nucleic acid molecule according to claim 3 which comprises a base sequence as depicted in SEQ ID No. 4 or a base sequence which only differs from the sequence as depicted in SEQ ID No. 4 because of the degeneracy of the genetic code.

7. A nucleic acid molecule according to claim 3 which only possesses a base sequence which is selected from the group consisting of SEQ ID No. 2, SEQ ID No. 3 and SEQ ID No. 4 group of base sequences or from a base sequence which only differs from one of said sequences because of the degeneracy of the genetic code.

8. A vector which comprises a nucleic acid molecule according to claim 1.

9. A vector according to claim 8 which is suitable for transforming a host cell.

10. A vector according to claim 9 in which the host cell is a microorganism.

11. A vector according to claim 10 in which the microorganism is a filamentous fungus.

12. A vector according to claim 11 in which the filamentous fungus is selected from the group consisting of Penicillium chrysogenum, Penicillium notatum, Penicillium brevicompactum, Penicillium citrinum, Acremonium chrysogenum, Aspergillus nidulans, Aspergillus niger, Aspergillus fumigates, Aspergillus ferrous and Tolypocladium inflatum.

13. A vector according to claim 12 in which the filamentous fungus is Penicillium chrysogenum.

14. A host cell which is transformed with a nucleic acid molecule according to claim 1.

15. A host cell according to claim 14 which is a microorganism.

16. A host cell according to claim 15 in which the microorganism is a filamentous fungus.

17. A host cell according to claim 16 in which the filamentous fungus is selected from the group consisting of Penicillium chrysogenum, Penicillium notatum, Penicillium brevicompactum, Penicillium citrinum, Acremonium chrysogenum, Aspergillus nidulans, Aspergillus niger, Aspergillus fumigates, Aspergillus ferrous and Tolypocladium inflatum.

18. A host cell according to claim 17 in which the filamentous fungus is Penicillium chrysogenum.

19. A process for producing penicillin which comprises culturing a host cell according to claim 18 under conditions which are suitable for bringing about the formation of penicillin by the host cell.

20. The process according to claim 19 in which the penicillin is penicillin G or penicillin V.

21. The process according to claim 19 which further comprises the isolation of the penicillin which has been formed.

22. An isolated protein which comprises an amino acid sequence as depicted in SEQ ID No. 1.

23. A protein according to claim 22 which only possesses the amino acid sequence as depicted in SEQ ID No. 1.

24. A host cell which is transformed with a vector according to claims 8.
